(19) United States
(12) Patent Application Publication
(10) Pub. No.: US 2003/0159169 A1
(43) Pub. Date: Aug. 21, 2003

(54) Cell culture system and methods of use

(75) Inventors: Paul Collodi, West Lafayette, IN (US); Lianchun Fan, West Lafayette, IN (US); Chunguang Ma, Hoffman Estates, IL (US)

Correspondence Address: MUETING, RAASCH & GEBHARDT, P.A., P.O. BOX 581415, MINNEAPOLIS, MN, 55458, US

(73) Assignee: Purdue Research Foundation, West Lafayette, IN

(21) Appl. No.: 10/319,221

(22) Filed: Dec. 13, 2002

Related U.S. Application Data
(60) Provisional application No. 60/341,355, filed on Dec. 13, 2001.

(30) Foreign Application Priority Data
Feb. 12, 2002 (CA) 2,371,460

(51) Int. Cl. A01K 67/027; C12N 5/06

(52) U.S. Cl. 800/20; 435/325

(57) ABSTRACT

The present invention is directed to methods of making a fish embryo cell line that can give rise to germ cells when introduced to a fish embryo. The present invention is further directed to methods of using the fish embryo cell line, making germ line chimeric fish, and to a cell culture medium.

### CONTINUING APPLICATION DATA

This application claims the benefit of U.S. Provisional Application Serial No. 60/341,355, filed Dec. 13, 2001, and Canadian Patent Application No. 2,371,460, filed Feb. 12, 2002, each of which are incorporated by reference herein.

### GOVERNMENT FUNDING

The present invention was made with government support under Grant No. 99-35205-8186, awarded by the United Department of Agriculture, and Grant No. R/MBE-06-98, awarded by the National Sea Grant Program. The Government has certain rights in this invention.

### BACKGROUND

The zebrafish is a popular model for studies of vertebrate development, possessing many favorable characteristics including a short generation time, external fertilization, and optically clear embryos that are suited for manipulations involving DNA transfer, cell labeling, and transplantation (Streisinger et al., Nature (London), 291, 293-296 (1981); Nusslein-Volhard, Science, 266, 572-574 (1994)). Also, methods have been established for performing large-scale mutagenesis screens in zebrafish to identify developmentally important genes (Drieveret al., Trends Genet., 10, 152-159 (1994); Mullins and Nusslein-Volhard. Curr. Opin. Genet. Dev., 3, 648-654 (1993); van Eeden et al., In Methods in Cell Biology, Detrich et al., (eds.) (Academic, San Diego), 21-41 (1999)). Despite these advantages, one deficiency of the zebrafish model is the absence of methods for targeted gene inactivation. In mice, the use of pluripotent embryonic stem (ES) cell cultures for the production of knockout mutants has provided a powerful approach for the study of gene function during embryogenesis. Cultured ES cells possess the ability to contribute to multiple tissues (including the germ line) after their introduction into a host embryo.

In mice, the use of pluripotent ES cell cultures for the production of specific mutants has provided a valuable approach to the study of gene function during embryogenesis and growth. The cultured ES cells possess the ability to contribute to multiple tissues (including the germ line) following their introduction into a host embryo. Gene inactivation is accomplished in the cell cultures by the targeted insertion of foreign DNA into the coding region of the gene by homologous recombination. ES cells that have undergone the targeting event are selected and grown in culture, and the genetic alteration is transferred to the germ line of a host embryo when the cultured cells are transplanted into the embryo, where they contribute to the germ-cell lineage (FIG. 1). A key to the successful employment of this approach is the derivation of embryo cell lines that are able to generate viable germ cells in vivo. In addition to pluripotent ES cell cultures, germ-line contribution can be accomplished by microinjecting host embryos with cultured primordial germ cells (PGC), the embryonic germ cell precursor. Although much effort has been devoted to the derivation of pluripotent ES cell and PGC lines from several species, continuously growing, multiple passage cultures that are able to contribute to the germ-cell lineage of a host embryo have been reported only for mice and chickens.

To use this cell-mediated strategy for the production of knockout mutants in fish, it is necessary to develop methods for the culture of cells that possess the ability to produce functional germ cells in vivo. Development of a cell-mediated approach to targeted gene inactivation for the zebrafish would greatly enhance the value of this organism as a model for the genetic analysis of vertebrate development. Primary and multiple-passage embryo cell cultures have been derived from zebrafish and medaka that exhibit in vitro characteristics that are associated with pluripotency including embryoid body formation, alkaline phosphatase activity and the ability to differentiate into multiple cell types in culture (Sun et al., Mol. Mar. Biol. Biotech., Vol. 4, pp. 193-199 (1995); Hong et al., Proc. Natl. Acad. Sci. USA, 95, 3679-3684 (1998); Ghosh et al., Cytotechnology, 23, 221-230 (1997); and Bradford et al., Mol. Mar. Biol. Biotech., 3, 78-86 (1994)). When introduced into host embryos, the cultured cells were able to participate in normal development and contribute to multiple tissues (Hong et al., Proc. Natl. Acad. Sci. USA, 95, 3679-3684 (1998); Speksnijder et al., Mol. Mar. Biol. Biotech., 6, 21-32 (1997)). Despite these encouraging results that indicate pluripotency, contribution of the cultured cells to the host embryo's germ line and the successful production of viable eggs or sperm has not been reported using multiple-passage cultures. The inability of the embryo cell cultures to generate germ line chimeras precludes their use for gene transfer experiments.

### SUMMARY OF THE INVENTION

The zebrafish possesses many favorable characteristics that make it a valuable model for genetic studies of human disease. Despite these advantages for genetic research, one deficiency of the zebrafish model has been the absence of methods for introducing targeted mutations present in a single cell into a zebrafish embryo such that the targeted mutation is present in germ cells in the resulting adult zebrafish. The present invention represents a pioneering advance in the art of making germ line chimeric zebrafish. Typically, in vitro incubation of cells obtained from a zebrafish embryo for as short as about 24 hours results in cells that cannot become functional germ cells when transplanted into a zebrafish embryo. A cell culture system has been developed that makes it possible to propagate in vitro cells obtained from a zebrafish embryo while preserving the cell's capacity to produce functional germ cells following transplantation into a zebrafish embryo. The embryo develops into a germ line chimeric zebrafish, where the transplanted cell is present in gonad tissues, and optionally other tissues, of the resulting germ line chimeric zebrafish, and the genetic material of the introduced cell can be inherited by offspring of the germ line chimeric zebrafish.

The present invention provides a fish embryo cell line. A cell of the fish embryo cell line is able to become a germ cell when introduced to a fish embryo, even after incubation in vitro for at least about 24 hours. Preferably, the fish embryo cell line is a zebrafish embryo cell line.

The present invention also provides a method for making a fish embryo cell line. The method includes providing an isolated fish cell, preferably an isolated zebrafish cell, obtained from a first fish embryo, and incubating the isolated fish cell in vitro for at least about 24 hours. The fish cell or progeny thereof have the ability to become a germ cell when introduced to a second fish embryo. The first fish embryo may be a gastrula-stage embryo or a blastula-stage embryo. The isolated fish cell may be incubated in vitro with a feeder layer, or with fish cell conditioned medium. The invention further includes an isolated fish embryo cell line obtained by the method.

The present invention provides a method for making a germ line chimeric fish. The method includes providing an isolated fish cell that has been incubated in vitro for at least about 24 hours, preferably in medium including a feeder layer or fish cell conditioned medium. The method further includes introducing the fish cell to a recipient fish embryo to result in a chimeric fish embryo, and incubating the chimeric fish embryo such that the embryo develops into a chimeric fish that includes a germ cell derived from the introduced fish cell. The fish embryo may be a blastula-stage embryo or a gastrula-stage embryo, preferably obtained from a zebrafish. The invention further includes a germ line chimeric fish and a chimeric embryo obtained by the method.

Also provided by the invention is a cell culture medium. The medium includes a growth factor and fish cell conditioned medium. The growth factor is either fibroblast growth factor or epidermal growth factor. Preferably, the fibroblast growth factor is human basic fibroblast growth factor, and preferably, the epidermal growth factor is mouse epidermal growth factor. Optionally, the medium includes both fibroblast growth factor and epidermal growth factor.

Another aspect of the invention includes a cell culture medium including a growth factor and a fish cell. The growth factor is fibroblast growth factor, preferably human basic fibroblast growth factor, or epidermal growth factor, preferably mouse epidermal growth factor. Optionally, the medium includes both fibroblast growth factor and epidermal growth factor.

### BRIEF DESCRIPTION OF THE FIGURES

FIG. 1. Cell-mediated gene transfer strategy using pluripotent ES cell cultures.

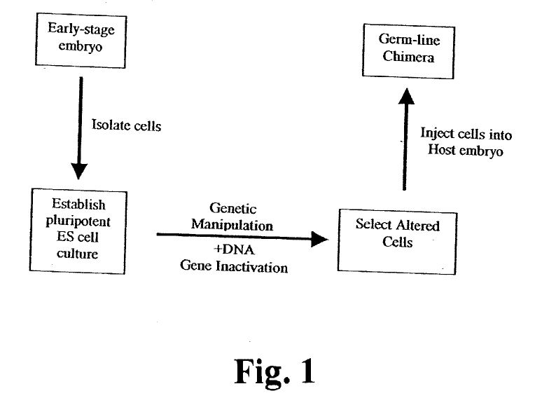

FIG. 2. Phase contrast photomicrographs of zebrafish embryo cell cultures. (a) Culture (24 hour) of embryo cell aggregates (arrow) on a feeder layer of RTS34st cells. (b) Culture (20 day old) of embryo cell aggregates (arrow) grown on an RTS34st feeder layer. (c) Culture (15 day old) maintained in the absence of feeder cells showing the presence of melanocytes (arrow). (d) Culture (10 day old) maintained in the absence of feeder cells showing the presence of neurites (arrow), indicating that neural cell differentiation has occurred. (e) Culture (25 day old) of embryo cells in RTS34st cell-conditioned medium without a feeder layer, illustrating embryo cell aggregates (arrow) on a monolayer of embryo fibroblasts.

FIG. 3. Distribution of vasa-positive embryo cells. Cultures maintained for 3 days (a) and 8 days (b) in RTS34st cell-conditioned medium or 3 days on RTS34st feeder cells (c) were examined by in situ hybridization by using a vasa-specific antisense probe. The control culture (d) was grown for 8 days in conditioned medium and hybridized with sense probe. [Magnification=×200 (a, b, and d), and=×100 (c).]

FIG. 4. Zebrafish phenotypes. (a) A germ line chimeric zebrafish from a GASSI embryo that had been injected at the blastula stage with cultured cells derived from B7-43 embryos. Melanocyte pigmentation is absent on the body of the chimera. (b) GASSI fish that was bred with the germ line chimera shown in a to produce (c) F1 individuals that exhibited a pigmentation pattern characteristic of B7-43 and (d) the nonpigmented GASSI phenotype.

FIG. 5. Contribution of ZEB cells to tissues of chimeric fish. Host GASSI blastulas were injected with ZEB cells (passage 5) and raised to sexual maturity. (A,B) DNA was isolated from tissues dissected from two adult fish and analyzed by PCR using EGFP specific primers designed to amplify a 481 bp product. The fish shown in A was known to be a germ-line chimera from F1 screening. (C) PCR analysis of tissues dissected from an F1 fish produced by breeding a founder chimera with a GASSI mate. All tissues examined from the F1 individual possessed EGFP sequences confirming germ-line contribution of the ZEB cells to the chimeric parent. Samples of liver and gonad were not obtained from the F1 individual and therefore not examined.

### DETAILED DESCRIPTION OF PREFERRED EMBODIMENTS OF THE INVENTION

The present invention includes fish embryo cell lines and methods for making fish embryo cell lines. As used herein, a “fish embryo cell line” is a culture of cells, derived from a fish embryo, in which the cells proliferate in vitro and do not display signs of morphological differentiation after, in increasing order of preference, at least about 1 day, at least about 5 days, at least about 14 days, most preferably, at least about 40 days culture in vitro. As used herein, in vitro refers to incubation of an isolated cell in tissue culture medium. As described in greater detail herein, cells of a fish embryo cell line are able to become germ cells when introduced to a fish embryo.

The method includes providing an isolated fish cell obtained from a fish embryo. As used herein, an “isolated” cell is a cell that has been physically separated from other cells to which it is attached in its natural environment. For instance, a fish embryo cell can be isolated from a fish embryo by physically separating the cells that make up the embryo. The method further includes incubating the isolated fish cell in vitro for, in increasing order of preference, at least about 24 hours, at least about 5 days, at least about 14 days, most preferably, at least about 40 days. In some aspects of the invention, the isolated fish cell is incubated with a second fish cell. Without intending to be limited by theory, the second fish cell may act as a feeder layer to preserve the ability of the fish embryo cells to become germ cells when introduced to a fish embryo, and/or inhibit the differentiation of the isolated embryo cells into, for instance, melanocytes or neuronal cells. Accordingly, the terms “second fish cell,” “feeder layer,” and “feeder cell” are used interchangeably. Unless otherwise specified, “a,” “an,” “the,” and “at least one” are used interchangeably and mean one or more than one.

The fish embryo used to obtain the isolated fish cell can be from, for instance, Danio rerio (also referred to herein as zebrafish), Oryzias latipes (medaka), or Oncorhynchus mykiss (Rainbow trout). Preferably, the fish embryo is from zebrafish. Any fish may be used as a donor of embryos for use in the methods described herein, and it is expected that the genotype of the fish cell isolated from a donor embryo will not have a significant impact, if any, on the ability of the isolated fish cell to become a germ cell when introduced to a fish embryo.

The fish embryo from which cells are derived may be at a stage of development where there are two or more cells present, preferably, the blastula-stage of development or the gastrula-stage of development, more preferably, the blastula-stage. The gastrula-stage of development is also referred to as the germ-ring stage of development. Determining if a fish embryo is at the blastula-stage or the gastrula-stage is routine to a person of skill in the art. Typically, a blastula-stage zebrafish embryo contains from about 128 cells to about 1,000 cells, preferably, about 1,000 cells. When the embryo is from a zebrafish, the blastula-stage typically occurs about 4 hours post-fertilization after incubation at 26° C. When the embryo is from a zebrafish, the gastrula-stage typically occurs about 6 hours post-fertilization after incubation at 26° C.

An embryo cell may be isolated from other cells of an embryo by methods that separate the cells of an embryo while maintaining their viability. Typically, embryos are collected at or before the appropriate developmental stage, rinsed, and, if necessary, incubated further until the appropriate developmental stage is reached. Preferably, an embryo is rinsed in a solution to kill bacteria without harming the embryo cells. Preferably, the solution contains bleach (for instance, sodium hypochlorite). Preferably, when bleach is used, the solution contains between about 0.05% and about 0.75% bleach, more preferably, about 0.5% bleach. Rinsing may also include alternately rinsing an embryo in a solution containing, for instance, bleach, followed by rinsing in water. Typically, the temperature of the rinse solutions are about 22° C.

After rinsing, and when the embryo is at the appropriate developmental stage, the embryo may be treated under conditions to remove the chorion surrounding each embryo. Methods for dechorionating a fish embryo are routine to a person of skill in the art, and include, for instance, exposure of the chorion to a proteinase, or manual removal using a forceps. Preferably, an embryo is exposed to a proteinase, preferably pronase. Optionally and preferably, the embryo is further exposed to a second proteinase treatment. Preferably, the second proteinase treatment includes trypsin, and optionally the second proteinase treatment includes a chelating agent, for instance, EDTA. Typically, the embryo is rinsed between the first and second proteinase treatments, preferably with a tissue culture medium that is compatible with fish cells. Preferably, the tissue culture medium is a mixture of Leibowitz's L-15, Dulbecco's modified Eagles, and Ham's F-12 media, (mixed in a ratio of about 50 parts Leibowitz's L-15, about 35 parts Dulbecco's modified Eagles, and about 15 parts Ham's F-12). Such a mixture is also referred to herein as LDF media. Preferably, the tissue culture medium is supplemented with about 10 nanomolar (nm) sodium selenite. The embryos are typically further manipulated to cause the embryo cells to be separated from each other. The resulting isolated fish embryo cells are ready to be placed in medium including a feeder layer.

The feeder layer used in the method may be obtained from a Teleost. Preferably, the feeder layer may be obtained from a Salmonid (for instance, a Rainbow trout), a Cyprinid (for instance, a zebrafish), or an Oryziinae (for instance, a medaka). Examples of the types of cells used for a feeder layer include, for instance, a stromal cell or a macrophage, preferably, a stromal cell. Whether a cell is a stromal cell can be determined by its ability to support the growth of hematopoietic cells, and such a determination is routine to a person of skill in the art. Examples of locations from which cells useful as feeder layers may be obtained include the spleen, blood, or head kidney, preferably, the spleen. Optionally and preferably, the cells of the feeder layer are immortalized or transformed, preferably, immortalized. Cells useful as feeder cells can be obtained using methods routine to a person of skill in the art. A preferred example of an immortalized feeder layer is the rainbow trout spleen stromal cell line designated RTS34st (see Ganassin and Bols, In Vitro Cell Dev. Bio. Anim., 35, 80-86 (1999)).

To prepare the feeder layer for addition of an isolated fish embryo cell, the feeder layer is typically propagated using appropriate conditions to form a nearly confluent or confluent monolayer in a culture plate. For instance, when the feeder layer is an RTS34st cell, the cells are present in media, preferably, LDF media supplemented with sodium selenite. Isolated fish embryo cells are placed in medium including a monolayer of the feeder layer. The medium containing the isolated fish embryo cells and the feeder layer is typically left undisturbed to permit attachment of the isolated fish embryo cells to the feeder layer. Preferably, the following components are added to the medium, where the amount of each component is the amount added to about 1.8 milliters (ml) of media: from about 130 microliters (μl) to about 170 μl, preferably, about 150 μl serum, preferably fetal bovine serum; from about 10 μl to about 20 μl, preferably, about 15 μl fish embryo extract, preferably, zebrafish embryo extract; from about 20 μl to about 40 μl, preferably, about 30 μl fish serum, preferably trout serum; from about 20 μl to about 40 μl, preferably, about 30 μl insulin, preferably bovine insulin; and from about 900 μl to about 1,000 μl, preferably, about 945 μl of fish cell conditioned medium, preferably RTS34st conditioned medium. Preferably, the medium to which these components are added is LDF medium supplemented with sodium selenite. The medium also contains a growth factor. For instance, the medium may contain from about 10 μl to about 20 μl, preferably about 15 μl of a 10 nanogram per microliter (ng/μl) stock solution of epidermal growth factor, preferably mouse epidermal growth factor; or from about 10 μl to about 20 μl, preferably about 15 μl of a 10 ng/μl stock solution of fibroblast growth factor, preferably human basic fibroblast growth factor. Preferably, the medium contains both epidermal growth factor and fibroblast growth factor. Without intending to be limited by theory, the growth factor(s) may act to preserve the ability of the fish embryo cells to become germ cells when introduced to a fish embryo, and/or inhibit the differentiation of the isolated embryo cells into, for instance, melanocytes or neuronal cells.

Fish embryo extract, preferably zebrafish embryo extract, may be prepared as described in Collodi and Barnes (Proc. Natl. Acad. Sci. USA, 87, 3498-3502 (1990)). Fish cell conditioned medium, preferably, RTS34st conditioned medium, may be prepared by adding fresh medium, for instance L-15 medium, supplemented with about 30% bovine serum, preferably fetal bovine serum, to a confluent culture of cells in a tissue culture flask. The medium is typically incubated with the cells for three to five days at a temperature appropriate for the cells (for instance, about 20° C. for RTS34st cells) and then removed, filter sterilized and stored at about 4° C. until it used. Fish cell conditioned medium is generally used within about 7 days.

Optionally, feeder layer cells may be treated to prevent growth of the feeder layer. Typically, growth arrested cells are metabolically active but do not divide. Methods for treating cells to prevent growth in in vitro culture include, for instance, irradiation of the cells and/or exposing cells to an antineoplastic. Radiation sources emitting gamma rays are preferred. Examples of antineoplastics include mitomycin C. Preferably, the cells are irradiated. The proper dosage of radiation or antineoplastic can vary depending on, for instance, the cell type; however, determining the appropriate conditions to arrest the growth of cells is routine in the art. When cells are RTS34st and irradiated, they are preferably exposed to about 30 RADS. When cells are RTS34st and exposed to mitomycin C, they are preferably exposed to about 10 μg/ml of the antineoplastic.

The culture containing the fish embryo cells and the feeder layer (either growth arrested or not) is incubated at a temperature that is appropriate for both the fish embryo cells and the feeder layer. For instance, when the fish embryo cell is a obtained from zebrafish and the feeder layer is a cell obtained from a Rainbow trout, preferably an RTS34st cell, the incubation temperature is about 21° C. to about 23° C., preferably, about 22° C. Typically, when the fish embryo cells are obtained from a gastrula-stage embryo, after several hours the fish embryo cells form aggregates of cells that are in close contact and take on a round shape. After at least about 3 days incubation, preferably, at least about 5 days incubation, aggregates are identified that do not display morphological indications of differentiation. Morphological indications of differentiation of embryo cells include, for instance, the appearance of cells having different shape, color, and/or size. Determining whether embryo cells display morphological indications of differentiation is routine to a person of skill in the art. Such aggregates also typically have a smooth surface, and the individual cells are difficult to discern. Typically, from about 30 to about 50 such aggregates are removed from the culture, combined, and partially dissociated by the addition of about 0.2% proteinase, preferably, trypsin, for about 2 minutes. After stopping the action of the proteinase, by adding, for instance, bovine serum, the suspended cells are collected by, for instance, centrifugation. The fish embryo cells are suspended in medium, preferably, LDF medium, supplemented with sodium selenite and added to a tissue culture plate containing a monolayer of the feeder layer, preferably, RTS34st cells. The cells are incubated for sufficient time, for instance, about 16 hours, to allow the fish embryo cells to attach, and the components described above (i.e., serum, preferably fetal bovine serum; fish embryo extract, preferably zebrafish embryo extract; fish serum, preferably trout serum; insulin, preferably bovine insulin; fish cell conditioned medium, preferably RTS34st conditioned medium; and a growth factor, for instance epidermal growth factor, preferably mouse epidermal growth factor, fibroblast growth factor, preferably human basic fibroblast growth factor, preferably both epidermal growth factor and fibroblast growth factor) are added to the culture. This culture is referred to as passage 1.

The passage 1 culture is typically incubated from about 5 to about 8 days. Those cultures containing aggregates of fish embryo cells that do not show morphological indications of differentiation are harvested by removing substantially all cells from the tissue culture plate, preferably, by addition of trypsin to the culture. The suspended cells are collected by, for instance, centrifugation, suspended in medium, preferably, LDF medium, supplemented with sodium selenite, and added to a tissue culture plate, for instance a 25 cm² flask, containing a monolayer of the feeder layer, preferably, RTS34st cells. The cells are incubated for sufficient time, for instance, about 5 hours, to allow the fish embryo cells to attach, and the components described above (i.e., serum, preferably fetal bovine serum; fish embryo extract, preferably, zebrafish embryo extract; fish serum, preferably trout serum; insulin, preferably bovine insulin; fish cell conditioned medium, preferably RTS34st conditioned medium, and either epidermal growth factor, preferably mouse epidermal growth factor, or fibroblast growth factor, preferably human basic fibroblast growth factor, preferably both epidermal growth factor and fibroblast growth factor) are added to the culture. This culture is referred to as passage 2.

After about 4 days to about 7 days incubation, the cells are harvested by removing substantially all cells from the tissue culture plate, preferably, by addition of trypsin to the culture, and divided into two tissue culture plates, for instance, two 25 cm² flasks. This process of passaging the cells is repeated about every 5 days as the monolayer becomes confluent. A fish embryo cell line that can be grown on a feeder layer and includes cells that do not display morphological indications of differentiation for at least about 1 days can be used in the methods described herein. Such a fish embryo cell line can be used to make a germ line chimeric fish after, in increasing order of preference, at least about 1 day, at least about 5 days, at least about 14 days, most preferably, at least about 40 days culture in vitro.

When the fish embryo cells are obtained from a blastula-stage embryo, the fish embryo cells may or may not form aggregates. Preferably, cultures forming aggregates are used. When no aggregates are formed, the cells derived from a blastula-stage embryo typically form a confluent monolayer of cells in the primary culture (passage 1). These monolayers of cells are evaluated for the presence of cells displaying morphological indications of differentiation, and typically those cultures that do not are used. When the cells derived from a blastula-stage embryo do form aggregates in the primary culture, there is typically no need to identify those aggregates that do not display morphological indications of differentiation. Typically, all aggregates are removed from the culture, combined, and partially dissociated by the addition of about 0.2% proteinase, preferably, trypsin, for about 2 minutes. After stopping the action of the proteinase, by adding, for instance, bovine serum, the suspended cells are collected by, for instance, centrifugation. The fish embryo cells are suspended in medium, preferably, LDF medium, supplemented with sodium selenite and added to a tissue culture plate containing a monolayer of the feeder layer, preferably, RTS34st cells. The cells are incubated for sufficient time, for instance, about 16 hours, to allow the fish embryo cells to attach, and the components described above (i.e., serum, preferably fetal bovine serum; fish embryo extract, preferably zebrafish embryo extract; fish serum, preferably trout serum; insulin, preferably bovine insulin; fish cell conditioned medium, preferably RTS34st conditioned medium; and a growth factor, for instance epidermal growth factor, preferably mouse epidermal growth factor, fibroblast growth factor, preferably human basic fibroblast growth factor, preferably both epidermal growth factor and fibroblast growth factor) are added to the culture. This culture is referred to as passage 1. Subsequent passage of the cells is as described above for gastula-derived embryo cells.

Alternatively, instead of placing an isolated fish embryo cell in medium including a feeder layer, the isolated fish embryo cell can be placed in medium supplemented with fish cell conditioned medium, where the fish cell conditioned medium is at a concentration of about 50%. The production of fish cell conditioned medium is routine to a person of skill in the art. Preferably, the fish cell conditioned medium is obtained using RTS34st cells. Isolated fish embryo cells may be incubated in medium supplemented with fish cell conditioned medium and later transferred to medium containing a feeder layer.

When a fish embryo cell line is to be used in the methods described herein, including methods for making a germ line chimeric fish, the fish embryo cells are typically separated from the cells of the feeder layer. This may be done by exploiting the ability of the cells of the feeder layer to more quickly adhere to a tissue culture plate. For instance, after harvesting substantially all cells in a culture and replating on an empty tissue culture plate, after about 15 minutes of incubation the cells of the feeder layer have adhered to the plate and most of the fish embryo cells have not. Thus, removal of the medium from the plate permits removal of the fish embryo cells from the feeder layer cells.

The present invention is also directed to a germ line chimeric fish and methods for making a germ line chimeric fish. As used herein, “chimeric” is used interchangeably with mosaic, and refers to a fish or fish embryo made up of at least two, preferably two, genetically distinct populations of cells. The method includes providing a fish embryo cell line prepared as disclosed herein, and introducing at least one cell of the fish embryo cell line to a fish embryo under conditions such that the embryo develops into a germ line chimeric fish, where a daughter cell of the introduced fish embryo cell is present as a germ cell in the chimeric fish, and can be inherited by progeny of the chimeric fish. The present invention is also directed to a chimeric fish obtained by the method.

The method for making a germ line chimeric fish includes providing a cell obtained from a fish embryo cell line as described herein, and introducing the cell to a recipient fish embryo. Preferably, the recipient fish embryo is a blastula-stage embryo. Preferably, the cell obtained from the fish embryo cell line and the recipient fish embryo to which it is introduced are the same species of fish, preferably, zebrafish. Methods for introducing cells to fish embryos are routine to a person of skill in the art, and include, for instance, injection, or cultured cell-embryo aggregation, preferably, injection. Preferably, from about 50 to about 200 cells obtained from a fish embryo cell line are introduced to a fish embryo by injection.

The recipient fish embryo containing the introduced fish cell is allowed to develop into a fish using methods that are routine to a person of skill in the art, and the resulting fish (the F0 generation) is assayed to determine if it is a germ line chimeric fish containing as a germ cell a daughter cell of the introduced fish cell.

A variety of known methods may be used for determining if the resulting fish is a germ line chimeric fish, and generally include assaying for some unique characteristic of the introduced fish embryo cell. For instance, if the introduced fish embryo cell line encodes a dominant phenotype, the presence of the phenotype in F1 generation fish, or progeny thereof, can be used to evaluate if the F0 fish is a germ line chimeric fish. If a unique nucleotide sequence is present in the introduced fish embryo cell and not present in the cells of the recipient embryo, techniques such as the polymerase chain reaction (PCR), Southern blot, or Northern blot may be used on tissue, for instance gonad tissue or an embryo, obtained from the resulting fish.

The present invention includes progeny of F0 germ line chimeric fish that possess germ cells containing genetic material donated by the introduced fish cell. Such progeny may be obtained by crossing different F0 fish with each other, or crossing F0 fish with other fish.

The present invention is also directed to a cell culture medium. The cell culture medium includes a growth factor. The growth factor may be epidermal growth factor, preferably, mouse epidermal growth factor. Alternatively, the growth factor may be fibroblast growth factor, preferably, human basic fibroblast growth factor. Preferably, the medium contains both epidermal growth factor and fibroblast growth factor.

The cell culture medium also contains fish cell conditioned medium. The fish cell medium may be obtained from a hematopoietic cell, including, for instance, a stromal cell or a macrophage, preferably, a stromal cell, more preferably, a fish spleen stromal cell. Such a cell may be obtained from a rainbow trout. Preferably, the fish cell conditioned medium is obtained from RTS34st. Alternatively, the medium also contains a fish cell. The fish cell may be obtained from a hematopoietic cell, including, for instance, a stromal cell or a macrophage, preferably, a stromal cell, more preferably, a fish spleen stromal cell. Such a cell may be obtained from a rainbow trout. Preferably, the fish cell is RTS34st.

The present invention is illustrated by the following examples. It is to be understood that the particular examples, materials, amounts, and procedures are to be interpreted broadly in accordance with the scope and spirit of the invention as set forth herein.

### EXAMPLE 1

### Derivation of Zebrafish Embryo Cell Lines

Methods have been developed for the derivation of cell lines initiated from blastula- and gastrula-stage zebrafish embryos and we have demonstrated that the cultures are able to generate viable germ cells in vivo following introduction into a host embryo. Cell lines initiated from gastrula-stage embryos have been growing continuously in culture for at least 14 passages (11 weeks) and at least approximately 48 population doublings. Cultures up to 14 days old have been used to generate zebrafish germ line chimeras. In addition to the gastrula-derived lines, cell lines that have been maintained for at least 3 passages have been derived from zebrafish blasula-stage embryos. The gastrula and blastula-derived cell lines currently growing in our laboratory were initiated from three strains of fish. The fish used to derive these lines include wild type zebrafish, a transgenic strain of zebrafish carrying the bacterial neo gene, and a transgenic line of fish that carry and express the enhanced green fluorescent protein (EGFP) under the control of the fish beta-actin promoter.

Initiation of Cultures from Gastrula-Stage Zebrafish Embryos

Zebrafish embryos were collected about 1 hour after fertilization and the debris was removed with a Pasteur pipet. Groups of approximately 200 embryos were washed two times with bleach solution prepared by mixing 0.1 ml fresh bleach stock solution with 170 mls water (stock solution: sodium hypochlorite, 6.0%; Clorox Company, Oakland, Calif.). For each wash, the embryos were soaked in the bleach solution for 5 minutes and then rinsed 2 times with water. After the final water rinse, the embryos were incubated in approximately 100 ml of water for 5 minutes and then soaked a second time in the bleach solution for 5 minutes followed by 3 to 5 more rinses with water. Following the bleach treatment, the embryos were placed in 100 ml water and allowed to develop to the gastrula-stage (approximately 6 hrs post-fertilization, at about 26° C.).

Before being used to initiate the cell cultures, the embryos were subjected to the following series of bleach treatments. For each treatment the bleach solution consisted of 40 ml water containing 0.2 ml fresh bleach. First, the embryos were soaked for 2 minutes in the bleach solution and then rinsed three times with water. Next the embryos were treated twice with bleach (90 seconds each treatment) and rinsed three times with water following each treatment. Finally, the embryos were treated with bleach one additional time (60 seconds) followed by three more water rinses. The embryos were then placed in 3 mls pronase solution (0.05% pronase, Sigma Co., St. Louis, Mo.) in 10% Hank's solution (GibcoBRL, Grand Island, N.Y.) and incubated for 30 minutes. After pronase treatment, the embryos were rinsed three times with LDF cell culture medium (a mixture of Leibowitz's L-15 (GibcoBRL), Dulbecco's modified Eagles (GibcoBRL), and Ham's F-12 media (GibcoBRL), (50:35:15), supplemented with 10 nm sodium selenite (Sigma Co.)). The pronase treatment removed the chorion surrounding each embryo. The dechorionated embryos were then placed in 3 mis of trypsin solution (0.2% trypsin (Sigma Co.) containing 1 mM EDTA (Sigma Co.) in PBS (GibcoBRL)) for 1 minute to dissociate the cells. The embryos were pipeted up and down approximately 5 times with a 10 ml pipet and then transferred to a 15 ml centrifuge tube and 4 ml of LDF cell culture medium was added. The dissociated embryo cells were then collected by centrifugation (500×g; 5 minutes) and the resulting cell pellet was re-suspended in 3.6 mls LDF medium. The cell suspension was prepared at room temperature.

To initiate cultures, the embryo cells were seeded onto a confluent monolayer of RTS34st cells contained in each well of a 6-well cell culture plate (35-mm diameter well). The RTS34st cells were propagated as described by Ganassin and Bols (In Vitro Cell Dev. Biol. Anim., 35, 80-86 (1999)). The embryo cell suspension was added to two wells (1.8 ml/well) of the 6-well plate, and the plate was left undisturbed for 30 minutes to allow the embryo cells to attach to the RTS34st monolayer. After the embryo cells attached, the following factors were added to each well: fetal bovine serum (FBS, 150 μl, Harlan, Indianapolis, Ind.), zebrafish embryo extract (15 μl, prepared as described in Collodi and Barnes, Proc. Natl. Acad. Sci. USA, 87, 3498-3502 (1990)), trout serum (30 μl, East Coast Biologicals, North Berwick, Me.), bovine insulin (30 μl of a 1 mg/ml stock solution, Sigma Co.), mouse epidermal growth factor (15 μl of a 10 ng/μl stock solution, GibcoBRL), human basic fibroblast growth factor (15 μl of a 10 ng/μl stock solution, GibcoBRL) and RTS34st conditioned medium (945 μl). The RTS34st conditioned medium was prepared by adding fresh L-15 medium (10 mls, Sigma Co.) supplemented with FBS (30%, Harlan, Indianapolis, Ind.) to a confluent culture of RTS34st cells contained in a 75 cm² tissue culture flask (Falcon, Franklin Lakes, N.J.). The medium was incubated with the cells for three to five days (20° C.) and then removed, filter sterilized and stored at 4° C. until it was used for embryo cell culture within a period of 7 days.

Several hours after adding the embryo cells to the RTS34st feeder layer, the embryo cells form tightly packed aggregates that were distributed throughout the culture. The cultures were incubated for 3 to 5 days (22° C.) to allow the embryo cells to proliferate and the cell aggregates to increase in size. The optimal temperature for the zebrafish embryo cells is 26° C. However since the RTS34st cells do not survive well at this temperature, the zebrafish embryo cell/RTS34st co-culture was maintained at 22° C. This temperature supported adequate growth of the embryo cells and was not lethal for RTS34st. At this time individual cell aggregates were identified and removed from the culture using a hollow needle formed from a drawn out Pasteur pipet. Approximately 30 to 50 aggregates, that consist of tightly packed cells and possess a homogeneous appearance with no morphological indication of differentiation, were selected. The selected cell aggregates were picked from the culture with the hollow needle and combined in a centrifuge tube. The aggregates were partially dissociated into smaller cell aggregates by adding 1 ml trypsin (0.2% containing 1 mM EDTA in PBS) and incubating 2 minutes. The cell suspension was pipetted up and down approximately 5 times through a 5 ml pipet, then 0.1 ml FBS was added to stop the action of the trypsin and the cells were collected by centrifugation (500×g, 5 minutes). The cells were suspended in 1.8 ml LDF cell culture medium and added to a single well of a 6-well plate containing a monolayer of RTS34st cells. The embryo cells were allowed to attach for 16 hours and then the same factors that are listed above were added to the well. At this point the culture consisted of many small embryo cell aggregates along with some individual cells attached to the RTS34st feeder layer. This culture is referred to as a passage 1 culture.

The culture was incubated for 5 to 8 days (26° C.) during which time the cells proliferated and the aggregates increased in size without showing morphological indications of differentiation. At this time, all of the cells were harvested by adding 2 mls of trypsin to the well and incubating 1 minute. The mixture of dissociated embryo and RTS34st cells was removed from the well and transferred to a centrifuge tube and 0.1 ml FBS was added to stop the action of the trypsin. The cells were collected by centrifugation (500×g, 5 minutes) and the cell pellet was suspended in 1.6 mls LDF cell culture medium. The cell suspension was 2 added to a 25 cm flask, containing a confluent monolayer of RTS34st cells and the embryo cells were allowed to attach to the feeder layer for 5 hours. The following factors were then added to the flask: FBS (300 μl), zebrafish embryo extract (30 μl), trout serum (60 μl), bovine insulin (60 μl of a 1 mg/ml stock), mouse epidermal growth factor (30 μl of a 10 ng/μl stock solution), human basic fibroblast growth factor (30 μl of a 10 ng/μl stock) and RTS34st conditioned medium (1,890 μl). The embryo cells formed aggregates on the feeder layer and increased in size as the embryo cells proliferate. This culture is referred to as a passage 2 culture.

After 4 to 7 days the cells were harvested from the flask by trypsinization as described above and the cell suspension was divided into two 25 cm² flasks. With each passage the cell aggregates became easier to dissociate so that by passage 4 or 5 the cell suspension consisted of single cells. At each passage the embryo cells initially formed small homogenous appearing aggregates that attached to the RTS34st feeder layer. The aggregates disappeared as the cells migrated out and grew into a monolayer after approximately 14 hours. The cells were passaged approximately once every 5 days as the monolayer became confluent.

The embryo cells possessed a long, thin, spindle-shaped morphology and grew to form dense bundles of tightly packed cells that were in close association with the RTS34st feeder layer. To separate the embryo cells from the RTS34st feeder layer, the cells were harvested by trypsinization as described above and the mixture of embryo and RTS34st cells were plated onto an empty tissue culture dish (100 mm diameter, Falcon, Franklin Lakes, N.J.). After 15 minutes the RTS34st cells began to attach and spread on the plastic surface while the embryo cells formed aggregates that were floating in the medium or loosely attached to the culture surface. The embryo cells were harvested by first removing and saving the medium contained in the flask and then gently pipeting fresh medium onto the culture surface. The embryo cell aggregates were released by the gentle pipeting leaving behind the RTS34st cells attached to the surface. The medium containing the released embryo cell aggregates was combined with the medium already removed from the flask and the embryo cells were collected by centrifugation.

Initiation of Cultures from Blastula-Stage Zebrafish Embryos

Cell cultures have been initiated from blastula-stage zebrafish embryos. To initiate the cultures, the embryos were treated as described above except that they were pronase-treated and dissociated at the blastula-stage of development (1000 cell stage, approximately 4 hours post-fertilization). Cells from the blastula-stage embryos were seeded onto a monolayer of RTS34st cells as described above. Unlike the gastrula-derived cultures, the cultures initiated from blastulas occasionally did not form cell aggregates on the feeder layer but proliferated to form a confluent monolayer of cells in the primary culture. In these cases, the blastula-derived cultures possessed a morphology that closely resembled the passage 5 and older gastrula cultures consisting of long spindle shaped cells that grew into dense bundles in close association with the RTS34st feeder layer.

### EXAMPLE 2

### Production of Zebrafish Germ-Line Chimeras from Embryo Cell Cultures

This Example demonstrates that an embryo cell culture is able to contribute to the germ line of a host embryo and the cultured cells produce viable germ cells (eggs or sperm) in the host.

Materials and Methods

Zebrafish Strains. Zebrafish of the transgenic B7-43 strain possess melanocyte pigmentation on their bodies and are homozygous for the bacterial neomycin phosphotransferase gene (neo; Gibbs et al., Mol. Mar. Biol. Biotechnol., 3, 317-326 (1994)). Zebrafish from the GASSI strain are homozygous for the b2 allele at the brass locus and therefore lack heavy melanocyte pigmentation on their bodies (Gibbs and Schmale, Mar. Biotechnol., 2, 107-125 (2002)).

Cell Culture. Zebrafish embryo cells were cultured for use as primordial germ cells/pluripotent embryonic stem cells as described in Examples 1. Embryos used for cell culture were obtained from the B7-43 line of zebrafish.

At various times after culture initiation, zebrafish embryo cells were harvested and microinjected into recipient embryos or examined for the presence of vasa mRNA, a primordial germ-cell marker. Cultures to be used for microinjection were initiated on RTS34st feeder layers as described above. To harvest the cells, EDTA solution (0.25 mM EDTA in PBS) was added to the culture, causing the embryo cells to dissociate from the feeder layer. The embryo cells were collected, leaving the RTS34st cells behind, and dissociated to single cells with gentle pipeting. After being washed one time, the cells were suspended in LDF medium and loaded into a needle formed from a drawn-out Pasteur pipet.

Embryo Microinjection. Recipient embryos used for cell transplantation experiments were obtained from the GASSI line of fish that lack heavy melanocyte pigmentation on the body. Blastula-stage embryos were dechorionated with pronase, rinsed in embryo medium (Westerfield, M. The Zebrafish Book: A Guide for the Laboratory Use of Zebrafish (Danio rerio) (Univ. of Oregon Press, Eugene, Oreg.), pp. 2.1-2.10 (1995)), and placed in a depression made in agarose in a 60-mm Petri dish. Approximately 50 to 200 cells were delivered into the cell mass of each recipient blastula by using a dissecting microscope and a hand-held Pipet-Aid (Sigma; ref. Lin et al., Proc. Natl. Acad. Sci. USA, 89, 4519-4523 (1992)). After injection (1 hour), the embryos were transferred into a Petri dish containing water; after 7 days, they were placed in a recirculating incubator system. After an additional 7 days, the embryos were transferred to a 2.5-gallon tank and reared to sexual maturity (approximately 3 months). To test for germ-line chimerism, individual fish developed from injected embryos were bred to GASSI fish and the F1 individuals were examined for the presence of neo or melanocyte pigmentation as described below.

Embryo Analysis. PCR analysis was performed on a group of the embryos at various times after injection and on embryos from the F1 generation. DNA extraction was performed by using the QIAamp DNA Mini Extraction kit (Qiagen, Chatsworth, Calif.), and neo sequences were detected with the following primers: forward, 5′-GGATGATC TGGACGAAGAGC (SEQ ID NO: 1) and reverse, 5′-GAAATCTC GTGATGGCAGGT (SEQ ID NO:2). The PCR reaction mixture contained genomic DNA (100 ng), Tris-HCl (10 MM, pH 8.3), KCl (50 mM), dNTPs (200 μM), and each primer (1 μM). Typical amplification conditions consisted of 35 cycles of 1 minute each at 94° C., 62° C., and 72° C. The 392-base pair amplification product was visualized by agarose-gel electrophoresis and ethidium-bromide staining. Product identity was confirmed by sequencing. Southern blot analysis was conducted on genomic DNA isolated from embryos, adult fish, and cultured cells (Sambrook et al., Molecular Cloning: A Laboratory Manual (Cold Spring Harbor Lab. Press, Plainview, N.Y.), pp. 9.31-9.62 (1989)). DNA (35 μg) was digested with EcoRV, fractionated by agarose-gel electrophoresis, and transferred to a nylon membrane (Schleicher & Schuell, Keene, N.H.). Hybridizations were conducted at 65° C. in a solution containing SSC (6×; 1×SSC=0.15 M sodium chloride/0.015 M sodium citrate, pH 7), Denhardt's solution (6×), SDS 0.1%, and yeast RNA (0.1 mg/μl) by using a ³²P-labeled probe generated by PCR amplification of neo sequences from the pBK-RSV plasmid (Stratagene). The probe was purified by using ProbeQuant G-50 micro columns (Amersham Pharmacia), and 2×10⁶ cpm/ml was added to each hybridization. After hybridization, the membranes were subjected to high-stringency washes (0.1 ×SSC and 0.1% SDS, 65° C.).

Detection of vasa mRNA. For reverse transcription-PCR analysis of embryo cell cultures, total RNA extraction and cDNA synthesis were performed by using the QuickPrep RNA Extraction kit (Amersham Pharmacia) and Smart cDNA Synthesis kit (CLONTECH), respectively. PCR was conducted by using the following primers: 5′-TGTGGACGTGAGTGGCAGCAATC (SEQ ID NO: 3) and 5′-CTAGATAGCGCACTTTACTCAGG (SEQ ID NO: 4) with 35 cycles at 94° C. (30 seconds), 64° C. (1 minute), and at 72° C. (1 minute); the 505-base pair product was visualized by agarose-gel electrophoresis. Product identity was confirmed by sequencing. For in situ hybridization, embryo cells cultured on a glass chamber slide (Nunc, Rochester, N.Y.) were fixed (4% formaldehyde, 5% acetic acid in 0.9% NaCl), dehydrated in a xylene series, rehydrated, treated with pepsin (0.1% pepsin, 10 min at 37° C.), and postfixed (1% formaldehyde in PBS) before hybridization (16 h at 42° C.; ref. Raap et al., In Methods in Molecular Biology, ed. Choo, K. H. A. (Humana, Totowa, N.J.), Vol. 33, pp. 293-300 (1994)) After being washed (at 50° C.), the cells were cleared with glycerol, the number of stained colonies was determined, and the diameter of each colony was measured at the widest point. The probe was prepared by in vitro transcription (MAXIscript; Ambion, Austin, Tex.) by using a 0.5-kb fragment of vasa cDNA as a template (nucleotides 779-1284, GenBank accession number AB005147; Yoon et al., Development (Cambridge, U.K.), 124, 3157-3165 (1997)).

Results

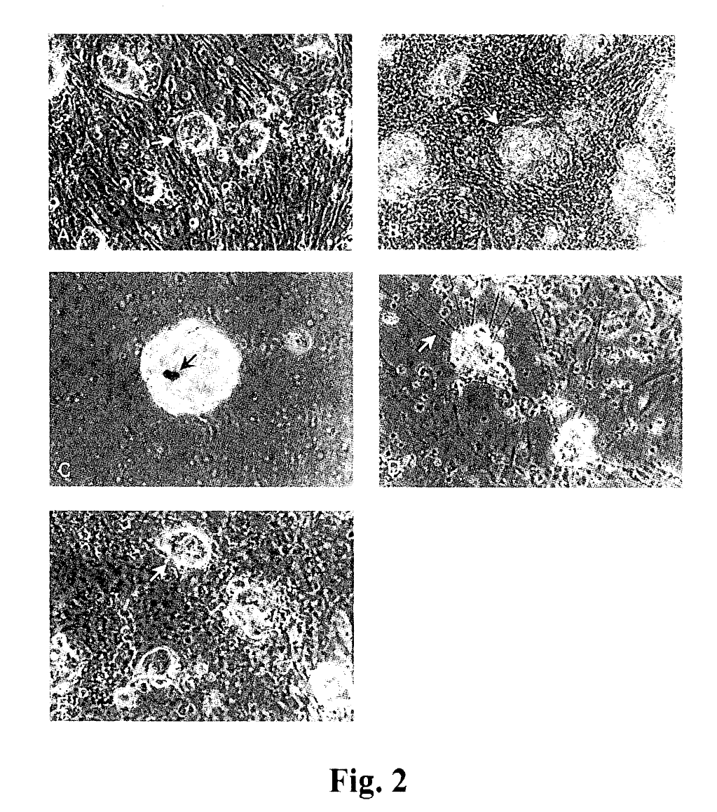

Zebrafish cells obtained from germ-ring stage embryos were cocultured on a monolayer of the RTS34st cell line (Ganassin and Bols, In Vitro Cell Dev. Biol. Anim., 35, 80-86 (1999); FIG. 2A). In these conditions, the embryo cells began to aggregate within 6 hours to form homogeneous clusters of tightly adherent cells distributed throughout the culture. In the presence of the feeder layer, the embryo cell aggregates continued to increase in size for more than 20 days without exhibiting morphological characteristics of differentiation (FIG. 2B). In contrast, embryo cells maintained in the same conditions without the RTS34st cells formed aggregates that exhibited characteristics of differentiated melanocytes and neuronal cells after 5 days in culture. Groups of pigmented melanocytes were apparent throughout the cultures and the neurites extended from the cell aggregates, making contact with neighboring cells (FIGS. 2C and 2D). Previous studies have shown that the neurites possess synaptic vessicles and growth cone morphology, and their appearance coincides with both elevated levels of acetylcholinesterase enzyme activity and the expression of neuron and astrocyte-specific marker proteins in the culture (Ghosh et al., Cytotechnology 23, 221-230 (1997)). Addition of RTS34st cell-conditioned medium to the embryo cell cultures delayed the appearance of the differentiated cell types until approximately day 25 (FIG. 2E).

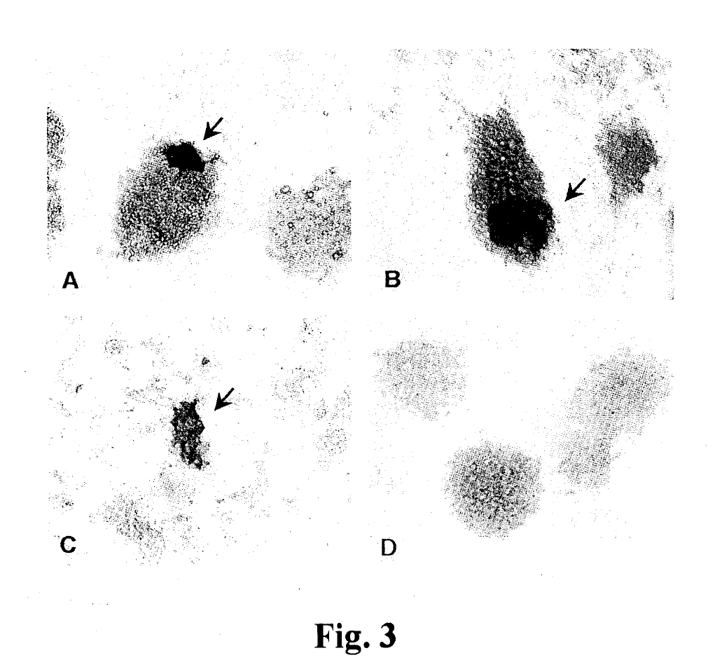

To determine whether the embryo cell cultures exhibited characteristics of primordial germ cells (PGCs), the cultures were evaluated by reverse transcription-PCR for the expression of the PGC marker gene, vasa (Knaut et al., J. Cell Biol., 149, 875-888 (2000), Braat et al., Dev. Dyn., 216, 153-167 (1999), and Yoon et al., Development (Cambridge, U.K.), 124, 3157-3165 (1997)). vasa mRNA was detected in embryo cell cultures maintained on the RTS34st feeder layers for at least 25 days. The vasa mRNA was also detected in 25-day-old cultures grown in the presence of RTS34st cell-conditioned medium in place of the feeder cells. Because the cultures maintained in the conditioned medium began to exhibit characteristics of differentiation around day 25, one of the cultures was passaged on day 24 by partially dissociating the embryo cell aggregates with trypsin and reseeding the small aggregates onto a feeder layer of RTS34st cells. Eight days after passage, the cell aggregates had increased in size without exhibiting signs of differentiation and continued to possess vasa mRNA. Removal of human leukemia-inhibitory factor (LIF) and stem cell factor from the culture medium did not affect the appearance or level of vasa mRNA when RTS34st cell-conditioned medium or feeder cells were present. In the absence of the feeder layer or cell-conditioned medium, vasa mRNA disappeared from the embryo cell cultures after 5 days. In the presence of cell-conditioned medium, the cultures produced a monolayer of fibroblastic cells along with the homogeneous cell aggregates that were distributed throughout the culture (FIG. 2E). In situ staining for vasa mRNA demonstrated that the vasa-positive cells were present within the aggregates (FIG. 3). The number and size of vasa-positive colonies increased over an 8-day culture period, indicating that the cells were proliferating. From day 4 to day 8, the number of vasa-positive colonies detected by in situ hybridization more than doubled (from 20 to 47), and the average size of the positive colony increased 25%.

As the spleen cells influenced the differentiation of the embryo cells in culture and promoted the survival of vasa-positive cells, the effect of the RTS34st feeder layers on the embryo cells ability to contribute to the germ-cell lineage of a host embryo was evaluated. Cell cultures, derived from the B7-43 transgenic line of zebrafish that possesses melanocyte pigmentation on the body and carries the bacterial gene, neo, were introduced by microinjection into host embryos obtained from the nontransgenic GASSI strain of zebrafish that lacks melanocyte body pigmentation (Gibbs et al., Mol. Mar. Biol. Biotechnol., 3, 317-326 (1994), and Gibbs and Schmale, Mar. Biotechnol., 2, 107-125 (2002)). Four groups of embryos were injected on separate days and the survival rate varied from 10% to 50%. PCR analysis of a group of embryos killed 3 weeks after each injection revealed that approximately 40% of the fish carried the neo gene, but only two individuals were identified that possessed melanocyte pigmentation on their bodies.

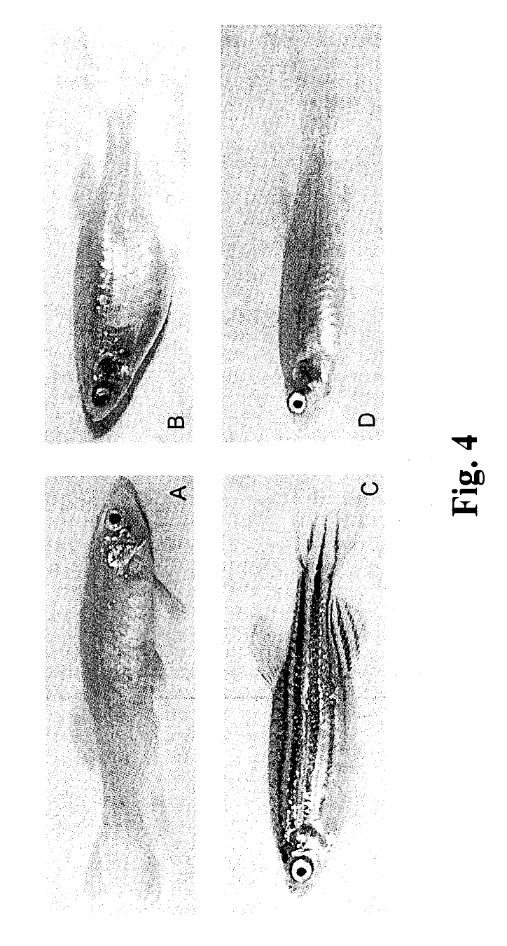

A total of 99 embryos that were injected with cells cultured on the RTS34st feeder layers survived to sexual maturity; four of these fish were found to be germ-line chimeras (Table 1). To identify the chimeras, each of the survivors was bred with a GASSI mate and the F1 individuals resulting from the cross were examined for the presence of neo and melanocyte pigmentation. Four fish, obtained from two different groups of injected embryos, produced neo-positive, pigmented (FIG. 4 and Table 1) F1 fish. Contribution of the cultured cells to the germ line in each of the four founder chimeras was variable, ranging from 1.2% to 8.2% of the F1 individuals who were neo positive and possessed pigmentation derived from the cultured cells (Table 1). The fish exhibiting the highest degree of germ-line chimerism (8.2%) was a female; the second highest individual (5.5%) was a male. None of the founder germ-line chimeras exhibited melanocyte pigmentation. Germ-line contribution was not detected when embryo cell cultures were maintained in the absence of the spleen cell feeder layers. To examine the stability of germ-line transmission, F2 generations were produced by breeding pigmented F1 sibling fish and by determining the frequency of F2 pigmentation and neo inheritance. As predicted from a cross involving two heterozygous F1 siblings, approximately 75% of the F2 fish were positive (Table 1).

TABLE 1
Frequency of germ-line chimerism.

| Group of | | | | |
|---|---|---|---|---|
| injected | | Germ-line | | |
| embryos¹ | Survivors² | chimeric³ | F1 transmission⁴ | F2 frequency⁵ |
| 1 | 49 | 1 | 19/342 | 198/267 |
| | | 1 | 1/82 | — |
| 2 | 16 | 0 | 0 | — |
| 3 | 26 | 1 | 1/81 | — |
| | | 1 | 8/97 | 93/126 |
| 4 | 8 | 0 | 0 | — |
| Total | 99 | 4 | 29 | — |

¹Each group of embryos was injected with cultured cells on different days.
²Injected embryos that survived to sexual maturity.
³Survivors were bred with GASSI fish and germ-line chimeras were identified by the production of neo⁺ pigmented F1 fish.
⁴Number of neo⁺ pigmented F1 fish produced by each germ-line chimera/total number of embryos produced.
⁵Number of neo⁺ pigmented F2 fish produced by breeding positive sibling fish from the F1 generation/total number of embryos produced.

Further PCR analysis of founder germ line chimeras revealed that the cultured embryo cells up to 14 days old were able to contribute extensively to host tissues. In addition to the gonad, neo sequences were detected in muscle, head, liver, skin, fin and gut indicating that the cultured embryo cells behave in vivo like pluripotent ES cells. The frequency of chimerism varied from 4 to 15%. Multiple passage embryo cell lines have been derived that were initiated and maintained in co-culture with the RTS34st cells. Cultures, established from zebrafish gastrula-stage embryos, have been maintained for at least 13 passages and approximately 50 population doublings on the RTS34st feeder layers. The growth rate of the cultures has remained constant and flow cytometric analysis has revealed that the cells are diploid. To facilitate in vivo analysis of germ line contribution, the cultures were derived from a transgenic line of fish that expresses the enhanced green fluorescent protein (EGFP) (Clontech, Palo Alto, Calif.). The presence of EGFP will make it possible to identify germ line chimeras by the production of fluorescent embryos in the F1 generation. In ongoing experiments, cultured cells at each passage are being injected into host embryos and the resulting fish are being examined for germ line chimerism as they become sexually mature.

Discussion

The results of this study indicate that factors released by RTS34st cells inhibit zebrafish embryo cell differentiation and enhance the survival of zebrafish primordial germ cells and/or pluripotent ES cells in culture. Although suppression of melanocyte and neuronal cell differentiation was observed in embryo cell cultures maintained on either RTS34st feeder cells or in cell-conditioned medium, a greater inhibitory effect was found in the presence of the feeder cells. A similar situation has been reported for mouse embryonic stem (ES) cells cultured in the presence of embryonic fibroblasts compared with fibroblast-conditioned medium (Smith et al., Dev. Biol., 151, 339-351 (1992)). Pluripotency of the cells was maintained more efficiently by the fibroblasts and was attributed to the presence of both the matrix-associated and the soluble forms of LIF in the cocultures, as opposed to only the soluble peptide in conditioned medium (Wells, In Transgenesis Techniques: Principles and Protocols, eds. Murphy, D. & Carter, D. A. (Humana, Totowa, N.J.), pp. 183-216 (1993), and Joly et al., Mol. Reprod. Dev., 53, 394-397 (1999)). LIF and stem cell factor are both required for the in vitro survival and growth of mammalian PGCs (Matsui et al., Cell, 70, 841-847 (1992)), but the addition of mammalian LIF and stem cell factor to the zebrafish culture medium did not affect the persistence of vasa mRNA in the embryo cell cultures. Presumably the fish equivalents of these peptides may be among the factors supplied by the RTS34st cell line. It has been demonstrated (Ganassin and Bols, In Vitro Cell Dev. Biol. Anim., 35, 80-86 (1999)) that RTS34st cells support the growth of trout macrophages in coculture, and that conditioned medium from RTS34st cells enhances [³H]thymidine incorporation by trout peripheral blood and head kidney leukocytes, indicating that the spleen cells produce and release soluble growth factors into the medium.

Embryo cells were able to contribute to the host germ line if cultured in the presence of RTS34st cells. The frequency of germ-line chimeras was low (4%) compared with published frequencies for mouse ES cells (5%-14%; Gossler et al., Proc. Natl. Acad. Sci. USA, 83, 9065-9069 (1986)), but this frequency is compensated for by the large number of fish embryos available for injection. Although the degree of germ-line chimerism in individual founder fish was also low, it was adequate for generating transgenic lines because relatively large numbers of embryos (30 to 100) are produced by a pair of fish at each spawning, and founder fish can be bred frequently (once per week; Westerfield, M. (1995) The Zebrafish Book: A Guide for the Laboratory Use of Zebrafish (Danio rerio) (Univ. of Oregon Press, Eugene, Oreg.), pp. 2.1-2.10 (1995)). In future work, contribution to the host germ line may be enhanced by using irradiated recipient embryos (Joly et al., Mol. Reprod. Dev. 53, 394-397 (1999)). Unlike the feeder layer, the conditioned medium was not able to preserve the embryo cell's ability to contribute to the host germ line for periods longer than 3 days in culture.

It may be possible to use cell-mediated gene transfer to target gene inactivation and generate zebrafish mutants. To accomplish this objective, longer-term cultures will be necessary to provide additional time for the introduction of the targeting vector and selection of colonies possessing the targeted insertion, as has been done with mouse cells (Capecchi, Science, 241, 1288-1293 (1989)). This production of long-term cultures will be aided by the fact that RTS34st cell-conditioned medium suppressed zebrafish embryo cell differentiation and promoted the in vitro propagation of vasa-positive cells.

### EXAMPLE 3

### Germ-Line Competent Zebrafish Cell Lines

This Example demonstrates that methods have been developed that support the continuous growth of zebrafish embryo cells in culture. Using these methods, the cultured embryo cells maintain the capacity to contribute to the germ line of a host embryo and generate the germ line chimeras for at least 6 weeks and 6 passages in culture.

Experimental Protocol

Zebrafish strains. A transgenic line of zebrafish homozygous for the EGFP gene under the control of the zebrafish β-actin promoter (Higashijima et al., Develop. Biol., 192, 289-299 (1997)) was used as a source of embryos for cell culture. Host embryos were obtained from the GASSI strain of fish that lack heavy melanocyte pigmentation on their bodies (Gibbs and Schmale, Mar. Biotech., 2, 107-125 (2000)).

Cell culture. RTS34st cells (obtained from N. Bols, University of Waterloo, Canada) were cultured (22° C.) as described (Ganassin and Bols, In Vitro Cell Dev. Biol. Animal., 35, 80-86 (1999)) in Leibowitz's L-15 medium (Sigma) supplemented with 30% FBS. Feeder layers were prepared by seeding the RTS34st cells at approximately 50% confluency in the appropriate culture vessel and allowing the culture to become confluent before adding the embryo cells. Growth-arrested feeder cells were prepared by irradiating the RTS34st cells with 30 RADS (Gammacell 200 Irradiator, Atomic Energy of Canada, Ltd.) before use. Conditioned medium was prepared by incubating fresh medium (Leibowitz's L-15 plus 30% FBS) on a confluent culture of RTS34st cells for 3 days. The conditioned medium was collected, filter sterilized and stored frozen (−20° C.) until use.

To initiate cell cultures from gastrula-stage embryos, approximately 50 germ-ring stage embryos (Kimmell et al., Developmental Dynamics, 203, 253-310 (1995)) were washed in bleach solution (0.5%), rinsed 3 to 5 times in LDF culture medium (Collodi, P. et al., Cell Biol. Tox., 8, 43-61 (1992)) and dechorionated in pronase (0.5 mg/ml pronase in Hanks solution). The embryos were dissociated in trypsin/EDTA solution (0.2% trypsin/1 mM EDTA in PBS) and the cells collected by centrifugation. The pellet was re-suspended in LDF medium and seeded into a single well of a 6-well culture plate (Nunc) containing a monolayer of RTS34st cells. The medium was supplemented with FBS (5%; Harlan Laboratories, Indianapolis, Ind.), trout serum (1%; East Coast Biologics, North Berwick, Me.), trout embryo extract (50 μg/ml) (Collodi and Barnes, Proc. Natl. Acad. Sci. USA, 87, 3498-3492 (1990)), bovine insulin (10 μg/ml; Sigma, St. Louis, Mo.), human epidermal growth factor (50 ng/ml; GIBCO) and human fibroblast growth factor (50 ng/ml; R & D Systems).

After approximately 5 days, colonies possessing an ES-like morphology were removed under sterile conditions using a drawn-out glass Pasteur pipet. The ES-like morphology included homogeneous appearing aggregates consisting of small, tightly packed cells showing no obvious morphological indications of differentiation. Approximately 30 to 50 colonies were combined in a tube, partially dissociated in trypsin/EDTA solution and after centrifugation, re-suspended in LDF medium. The cell suspension, consisting of small cell aggregates and some individual cells was added to a single well of a 6-well plate containing a confluent monolayer of RTS34st cells. The embryo cells were allowed to attach to the feeder layer for approximately 5 hrs before the supplements described above (5% FBS, 1% trout serum, 50 μg/ml trout embryo extract, 10 μg/ml bovine insulin, 50 ng/ml human epidermal growth factor, and 50 ng/ml human fibroblast growth factor) were added. The culture was incubated (23° C.) for 5 to 7 days before all of the embryo cell aggregates contained in one well were harvested and partially dissociated in trypsin/EDTA solution and passaged into 2 wells of a 6-well plate. After 5 days the cells contained in the two wells were combined and passaged into a 25 cm² flask (Falcon) containing a monolayer of RTS34st cells. By the 4^{th} passage the culture contained very few cell aggregates and the cells proliferated as a monolayer. This cell line was designated ZEG, and was typically passaged after about 5-7 days in culture.

Cultures were initiated from blastula-stage embryos as described for the gastrula-derived cultures except that the embryo cells were plated on growth-arrested irradiated (30 RADS) RTS34st feeder cells. After approximately 5 days all of the colonies contained in the primary culture possessed an ES-like morphology and were harvested by trypsinization and passaged. This cell line was designated ZEB, and was typically passaged after about 5-7 days in culture.

Embryo microinjection. Cultured cells were transplanted into host embryos obtained from the GASSI strain of zebrafish (Gibbs and Schmale, Mar. Biotechnol., 2, 107-125 (2002)) that lack heavy melanocyte pigmentation on the body. Recipient embryos at the blastula-stage of development (1000 cells) were dechorionated with pronase (Westerfield, M. The Zebrafish Book: A Guide for the Laboratory Use of Zebrafish (Danio rerio) (Univ. of Oregon Press, Eugene, Oreg.), p. 4.1 (1995)), rinsed with water and placed in a shallow depression made in agarose contained in a Petri dish (60 mm). Approximately 50 to 100 cells suspended in LDF medium were delivered into the cell mass of each recipient blastula by using a dissecting microscope and a drawn-out glass Pasteur pipet connected to a hand-held Pipet-Aid (VWR) (Lin et al., Proc. Natl. Acad. Sci. USA, 89, 4519-4523 (1992)) The injected embryos were allowed to recover for 1 hour before being moved to a Petri dish containing water. After 7 days the embryos were transferred to a finger bowl and after an additional 7 days into a 2.5-gallon tank. To test for germ-line chimerism, individual fish developed from the injected embryos were bred to GASSI fish and the F1 individuals were examined for the formation of melanocyte pigmentation or the expression of the EGFP marker gene by fluorescence microscopy. PCR assays were conducted to detect the presence of EGFP sequences in DNA isolated from tissues of adult chimeric fish. The PCR reaction contained 100 ng of genomic DNA, Tris-HCl (10 mM, pH 8.3), KCl (50 mM), dNTPs (200 μM), and each primer (1 μM, 5′-ACCCTGAAGTTCATCTGCACC (SEQ ID NO:5) and 5′-GTGCTCAGGTAGTGGTTGTC (SEQ ID NO:6).

Results and discussion

Derivation of a cell line from zebrafish gastrula-stage embryos. The ZEG cell line was initiated from zebrafish gastrula-stage embryos on a feeder layer of rainbow trout spleen cells (RTS34st) (Ganassin and Bols, In Vitro Cell Dev. Bio. Anim., 35, 80-86 (1999)). After five days, colonies that possessed an ES-like morphology, characterized by homogeneous clusters of tightly adherent cells, were individually removed from the primary culture, combined and partially dissociated. The resulting suspension of small cell aggregates was re-plated onto a fresh RTS34st monolayer (passage 1). The embryo cells were allowed to proliferate for approximately 5 days during which time the aggregates became larger while maintaining an ES-like morphology. All of the aggregates were then harvested in trypsin and re-seeded onto a fresh monolayer of RTS34st. With each passage, the cell aggregates became easier to dissociate so that by passage 4 a suspension of single cells was obtained. The ZEG cells possessed a fibroblast-like morphology and grew to form dense bundles of tightly packed cells on top of the feeder layer. To determine if the cells maintain the ability to contribute to the germ cell lineage of a host embryo, passage 6 ZEG cultures (6 weeks old) derived from zebrafish that possess wild-type pigmentation were injected into host embryos from the GASSI line of fish that lack melanocytes (Gibbs and Schmale, Mar. Biotechnol., 2, 107-125 (2002)). Surviving embryos were raised to sexual maturity and crossed with noninjected GASSI mates. Two germ-line chimeras were identified from approximately 90 fish that were screened. The germ-line chimeras were identified by the production of F1 embryos that possessed body pigmentation derived from the injected cells.

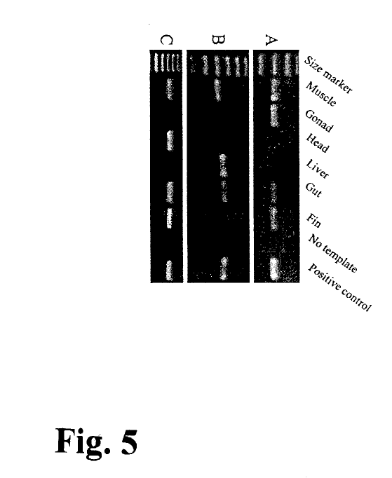

Derivation of a cell line from blastula-stage zebrafish embryos. A second embryo cell line (ZEB), was initiated from mid-blastula-stage embryos obtained from a transgenic line of fish that express the enhanced green fluorescent protein (EGFP) and possess wild-type pigmentation (Higashijima et al., Develop. Biol., 192, 289-299 (1997)). In contrast to ZEG, all of the ZEB cell aggregates in the primary culture possessed an ES-like morphology on the RTS34st feeder cells making it unnecessary to isolate individual colonies. After 5 days, all of the ZEB cell aggregates were harvested by trypsinization and re-seeded onto a fresh feeder layer. As with the ZEG culture, the ZEB cell aggregates became easier to dissociate with each passage, eventually proliferating as a monolayer by passage 4. The ZEB cultures consisted of large epithelial-like cells that reached confluency at a low density and expressed EGFP. To evaluate ZEB cells' ability to contribute to the germ cell lineage in vivo, cultures at passage 5 (4 weeks old) were injected into GASSI host embryos. Three days after injection, potential germ-line chimeras were identified by the presence of EGFP⁺ cells in the region of the gonad. Approximately 1% of the injected embryos were identified as potential germ-line chimeras in this manner. Five of the identified embryos were raised to sexual maturity and 2 were confirmed to be germ-line chimeras by the production of F1 embryos that possessed melanocyte pigmentation and expressed EGFP. PCR analysis of tissues taken from adult founder germ-line chimeras revealed that ZEB cells contributed to multiple tissues of the host embryo (FIGS. 5A,B). In addition to the gonad, EGFP sequences were detected in muscle, liver, gut, and fin indicating that ZEB behave in vivo as pluripotent ES cells. As expected, all of the tissues obtained from F1 fish produced by founder chimeras possessed EGFP sequences (FIG. 5C) further confirming germ-line contribution of the cultured cells. Similar results were obtained with the ZEG cultures.

This paper describes the first available zebrafish cell lines that remain germ-line competent for a sufficient amount of time in culture to allow in vitro genetic manipulation and selection. Along with the mouse, the zebrafish is the second species from which such cultures are available. It is noteworthy that in addition to using non-differentiated blastulas, germ-line competent cultures were also derived from gastrula-stage embryos by selecting colonies of ES-like cells. Both cell lines have been maintained in culture for at least 30 passages. The use of ZEB cells containing a fluorescent marker, EGFP, made it possible to efficiently identify the germ-line chimeras 2 days after injection without the need to conduct PCR assays. Forty-percent of the embryos identified by the presence of EGFP-expressing cells in the gonad were later confirmed to be germ-line chimeras. This visual screening method makes it possible to rapidly examine a large number of embryos soon after injection and identify the potential chimeras, significantly reducing the number of fish that must be raised to sexual maturity for F1 screening.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A fish embryo cell line, wherein a cell of the fish embryo cell line, after incubation in vitro for at least about 24 hours, will become a germ cell when introduced to a fish embryo.

2. The fish embryo cell line of claim 1 wherein the fish embryo cell line is a zebrafish embryo cell line.

3. A method for making a fish embryo cell line, the method comprising:
providing an isolated fish cell obtained from a first fish embryo;
incubating the isolated fish cell in vitro for at least about 24 hours, wherein the fish cell or progeny thereof become a germ cell when introduced to a second fish embryo.

4. The method of claim 3 wherein the first fish embryo is a gastrula-stage embryo or a blastula-stage embryo.

5. The method of claim 3 wherein the first fish embryo and second fish embryo are zebrafish embryos.

6. The method of claim 3 wherein the isolated fish cell is incubated in vitro with a feeder layer.

7. The method of claim 3 wherein the feeder cell is a stromal cell.

8. The method of claim 7 wherein the stromal cell is obtained from rainbow trout.

9. The method of claim 1 wherein the medium comprises fish cell conditioned medium.

10. The method of claim 9 wherein the fish cell conditioned medium is obtained from a stromal cell.

11. The method of claim 10 wherein the stromal cell is obtained from rainbow trout.

12. An isolated fish embryo cell line obtained by the method of claim 3.

13. A method for making a germ line chimeric fish, the method comprising:
providing an isolated fish cell that has been incubated in vitro for at least about 24 hours;
introducing the fish cell to a recipient fish embryo to result in a chimeric fish embryo; and
incubating the chimeric fish embryo such that the embryo develops into a chimeric fish comprising a germ cell derived from the introduced fish cell.

14. The method of claim 13 wherein the fish embryo is a blastula-stage embryo or a gastrula-stage embryo.

15. The method of claim 13 wherein the fish cell is a zebrafish cell and the fish embryo is a zebrafish embryo.

16. The method of claim 13 wherein the fish embryo cell of the fish embryo cell line is propagated in medium comprising a feeder layer or fish cell conditioned medium.

17. A germ line chimeric fish obtained by the method of claim 13.

18. A cell culture medium comprising a growth factor and fish cell conditioned medium, wherein the growth factor is fibroblast growth factor or epidermal growth factor.

19. The medium of claim 18 wherein the fish cell conditioned medium is obtained from a fish stromal cell.

20. The medium of claim 19 wherein the fish stromal cell is obtained from rainbow trout.

21. The medium of claim 18 wherein the fibroblast growth factor is human basic fibroblast growth factor.

22. The medium of claim 18 wherein the epidermal growth factor is mouse epidermal growth factor.

23. The medium of claim 18 wherein the medium comprises fibroblast growth factor and epidermal growth factor.

24. A cell culture medium comprising a growth factor and a fish cell, wherein the growth factor is fibroblast growth factor or epidermal growth factor.

25. The medium of claim 24 wherein the fish cell is a fish stromal cell.

26. The medium of claim 25 wherein the fish stromal cell is obtained from rainbow trout.

27. The medium of claim 24 wherein the fibroblast growth factor is human basic fibroblast growth factor.

28. The medium of claim 24 wherein the epidermal growth factor is mouse epidermal growth factor.

29. The medium of claim 24 wherein the medium comprises both fibroblast growth factor and epidermal growth factor.

30. A cell line having the characteristics of a cell line designated ZEB.

31. A cell line having the characteristics of a cell line designated ZEG.
